(19) United States
(12) Patent Application Publication
(10) Pub. No.: US 2021/0137735 A1
(43) Pub. Date: May 13, 2021

(54) APPARATUS AND METHOD OF TREATING BLEPHARITIS, MEIBOMIAN GLAND DYSFUNCTION AND DRY EYE DISEASE

(71) Applicant: Danelli Ocular Creations, LLC, Granger, IN (US)

(72) Inventors: Bryan Minelli, Rosamond, CA (US); Marco Minelli, Mishawaka, IN (US); Kevin Danahey, Granger, IN (US)

(21) Appl. No.: 17/096,618

(22) Filed: Nov. 12, 2020

Related U.S. Application Data
(60) Provisional application No. 62/934,306, filed on Nov. 12, 2019.

(51) Int. Cl. A61F 9/007; A46B 5/04

(52) U.S. Cl. CPC A61F 9/00709; A46B 2200/1006; A46B 5/04

(57) ABSTRACT

An apparatus and method are provided for treating blepharitis, MGD and DED. The apparatus is self-administered for removing debris from an eyelid. The apparatus includes bristles and a pair of pads formed within the apparatus and specifically within an eyelid contact region. When self-compressed against the eyelid of the user, the user can self-treat the eyelid border by exfoliating the meibomian glands to promote the clear flow of meibum onto the eye.

### CROSS-REFERENCE TO RELATED APPLICATIONS

This application incorporates by reference and claims the benefit of priority to U.S. Provisional Application 62/934,306 filed on Nov. 12, 2019.

### BACKGROUND

The present disclosure relates to an apparatus and method for the treatment of Dry Eye Disease (DED). More particularly, the present disclosure relates to an apparatus, and method for treating Meibomian Gland Dysfunction (MGD) and blepharitis, two of the most commonly associated causative factors in DED using a self-administered tool for removing debris from an eyelid.

DED is now considered a major global health concern. The prevalence of DED is increasing and estimates show that approximately 30 million people in the United States alone are affected with this condition. This disease is expected to increase, and will create a significant societal and economic burden on our healthcare system in the United States and globally. Factors such as increasing age, the use of digital devices, increases in chronic metabolic diseases such as diabetes, hypertension, autoimmune diseases, cancer, and the medications used to treat them all play a causative role. The use of contact lenses as well as environmental factors and hormonal changes also play a significant role. DED has a significant impact on quality of life and is a main contributing factor for loss of productivity in the workplace. DED causes difficulties with everyday activities such as computer use, reading, driving, and watching television. Pharmaceutical treatments for this disease are available, however these medications are very expensive and create an enormous economic burden for insurance companies and patients while creating a major barrier to access for the millions of patients who suffer from this disease. These medications also do not address the most common etiology of DED, MGD and blepharitis.

Recent clinical studies have shown that the most common cause of DED is from evaporative disease and MGD is the primary cause of this condition. It has been estimated that up to 86% of patients with DED have MGD. Since most patients with DED have MGD, it seems reasonable to question why significant dollars are spent on treatments that do not address the root cause of this disease. It is therefore imperative to create affordable innovative solutions for this disease while decreasing the socioeconomic burden of DED and improving the quality of lives of the patients who are afflicted with this condition.

Meibomian glands are sebaceous glands that are located on the lid margin of both the upper and lower eyelids. There are approximately 25 to 40 meibomian glands in the upper eyelid and 25-30 in the lower eyelid. Meibomian glands are responsible for the production of the outermost layer of the tear film. This layer consists of fatty acids and lipids known as meibum. This oily layer provides an important role in the function of the visual system. This protective layer of lipids prevents the evaporation of the tear film from the ocular surface and is necessary for optimal visual acuity. Failure to maintain an optimal lipid layer results in rapid evaporation of the tear film from the ocular surface resulting in blurred and fluctuating vision as well as symptoms such as burning, tearing, itching, photophobia, and infection.

MGD is a multifactorial disease that can cause the meibum to become thickened over time which ultimately causes the glands to become blocked, plugged, and obstructed. If left untreated, MGD will cause permanent atrophy, gland dropout, and irreversible loss of the meibomian glands.

Blepharitis is another condition strongly associated with MGD. Blepharitis is a condition that causes inflammation of the eyelids and eyelid margins. The three main causes of blepharitis are staphylococcal, seborrheic, and a parasite known as demodex. Studies show that up to 50% of the adult population has some form of blepharitis and recent studies show that 100% of patients over 70 have demodex blepharitis. The inflammation from these forms of blepharitis can lead to a cascade of events that ultimately create the presence of a biofilm on the lid margin. This biofilm and keratinization of the eyelid margin further lead to obstruction of the meibomian glands contributing to gland atrophy and permanent loss of the glands. Blepharitis, MGD, and DED all have overlapping symptoms such as blurred vision, tearing, itching, a sandy gritty feeling, and light sensitivity. These symptoms are so closely tied together that many experts and leaders in the industry are calling blepharitis, MGD, and DED one term known as Dry Eye Blepharitis Syndrome (DEBS) a catchall phrase of DED and its progeny or genus.

Now that research has uncovered major root causes of DED, it is incumbent upon clinicians, researchers, and innovators to address MGD and blepharitis and create treatment strategies that address both of these conditions concurrently. In order for treatments to be effective, innovative solutions should be created that focus on the four contributing factors of DED: (1) obstruction of the meibomian glands, (2) removal of the biofilm on the eyelids and eyelid margins, (3) reducing inflammation, and (4) stabilization of the tear film.

One of the most important aspects of treating DED is with patient self-administered, at-home treatments. These at-home treatments are very important for maintaining the integrity of the ocular surface by removing debris, scurf, collerattes, microorganisms, and keratinized skin that can accumulate on the base of the eyelashes and lid margin. It is this debris that is associated with the formation of the biofilm along the lid margins and the subsequent obstruction of the meibomian gland orifices. The blockage of these glands leads to atrophy and permanent gland loss, and progression of the disease and its symptoms.

The dental model for eye care is an emerging trend as daily removal of the biofilm can prevent DED. This model is analogous to brushing your teeth daily in order to remove plaque and prevent periodontal diseases of the teeth and gums. The dental model uses an in office deep cleaning every 6 months to one year along with daily self-administered teeth brushing and regular flossing. The eyecare model is similar with at home daily cleaning of the eyelids and lashes and more comprehensive in office procedures that could be performed by an eye care professional on a regular basis. Such in office procedures are intended to perform a deep cleaning of the lid margins and others to express the meibomian glands. The goals of these procedures are to improve the structure and function of the meibomian glands.

Many traditional and emerging treatments are available for at-home self-administration treatment and management of DED. As will be outlined below, they each have significant disadvantages to the simple, safe, affordable, and highly effective solution provided herein. Many of the current treatment strategies for at-home use are ineffective, time consuming, expensive, and create compliance issues.

One of the most common and traditional approaches for at home therapy for DED are the use of warm compresses, eyelid massage, and eyelid scrubs. Eyelid, scrubs can be solutions, foams, gels, sprays, and commercially available pads.

Warm compresses do play an important role in heating and melting the meibum within the gland channel helping to restore the obstruction. They do, however, have significant limitations as the heat has to travel through tissue, muscle, and fat in order to create enough heat to effectively melt the meibum to a sufficient level. In addition, the use of warm compresses do not massage the glands in order to express the blocked, plugged, or obstructions within the channel of the meibomian glands. Warm compresses can be time consuming, so patient compliance is a major issue.

Eyelid scrubs with baby shampoo and more recent commercially available preparations also play a role in current treatment, however, there are limitations with these home hygienic treatments. Compliance, improper technique, inability of some patients to properly touch the lid margin can lead to drop out. Although these do play an important role in maintaining the health of the lid margin, they all have one major limitation. None of these non-electrical at home products are effective at exfoliating the base of the eyelid margin where the eyelashes insert into this mucocutaneous margin. It is at this precise margin where microorganisms, debris, and biofilm are located and can be very difficult to remove without the use of an electronic device. Such devices can perform a deep cleaning, but these are most often performed in an office setting by an eye care professional. The at home foams, sprays, and gels used to treat blepharitis are applied with a fingertip, Q-tip, washcloth, pad, or cotton swab. Although these are important, none of the methods are highly effective at performing the much needed exfoliation of the lid margin. Using the following analogy, just as toothpaste plays a vital role in dental health, using a finger, washcloth, or cotton tip applicator to brush one's teeth on a daily basis would be very ineffective for removing plaque.

Topical antibiotics and steroid combinations have also been widely used in the treatment of blepharitis and MGD. These treatments however are not without significant side effects. Long term use of these pharmaceuticals can cause antibiotic resistance as well as glaucoma and the formation of cataracts. This causes further economic impact as surgery may be necessary to remove the cataracts caused by the steroid drops or ointments. Oral antibiotics such as doxycycline have been widely used and do have a role in addressing the inflammation associated with MGD, however there can be significant side effects such as photosensitivity, intestinal distress, and can also affect the health of the intestinal microbiome.

Another treatment for DED (including DEBS) is blephex. This small handheld electromechanical device perfomis a microblepharexfoliation of the eyelid margins using disposable microsponges. This effective treatment is useful for removal of the biofilm and keratinization of the lid margin. This procedure is a deep cleaning of the eyelid margin that is performed in an office setting by an eye care professional and is ideally repeated every six months or yearly. It is unable to be self-administered by the patient, as it needs to be performed by a trained technician, certified technician, or physician.

A more recent at home device used to treat blepharitis is NuLids. This hand held electrical device has removable silicone tip brushes. The brushes oscillate much like a handheld electric toothbrush and is indicated for treating blepharitis. Although this is a reasonable solution and has been shown in studies to be effective, it's expensive. This device is cost prohibitive for many patients and requires the user to purchase reusable heads on a regular basis which can be costly over the long term. Because of the costs associated with this device, this creates a barrier to entry for the many patients who suffer from this debilitating condition.

A need, therefore, exists for an at-home self-administered treatment brush that is simple to use, safe, affordable for patients, and highly effective at removing the debris and biofilm from the eyelid margin. If left untreated, this can lead to a cascade of events causing DED (including DEBS), uncomfortable symptoms, infections, loss of vision, and a decrease in quality of life. The use of an inexpensive self-administered, self-treatment apparatus or tool to remove debris from an eyelid on a regular basis and therefore be more effective than treatments in an office setting by an eye care professional utilizing an expensive and difficult to apply apparatus. Making a self-administered treatment tool inexpensive and readily available to a user not only encourages regular preventative treatment, but also helps maintain an eyelid margin where, if untreated, can lead to significant decrease in quality of life.

### BRIEF SUMMARY OF THE DISCLOSURE

The present disclosure provide an apparatus and method for treating blepharitis, MGD, DEBS and DED by simply and affordably removing debris from an eyelid of a user. The apparatus can be self-administered by the user in the home, and therefore is more readily used on a more frequent basis than if the user were to be treated by an eye care professional in an office setting using a more expensive apparatus and procedure.

The apparatus used herein is self-administered at-home, for example, by that user on himself or herself and comprises all of the needed components to remove debris from the eyelid, including the eyelid margin and eyelashes of the user. Debris includes any organic or inorganic material which can cause blockage or obstruction of the meibomian glands in the upper or lower eyelids. Removing debris from the eyelid and eyelashes therefore promotes proper production from the Meibomian glands to prevent blepharitis, MGD, DED and DEBS. Regular treatment from the present apparatus, and promoting inexpensive availability thereof, minimizes obstruction of the Meibomian gland while removing biofilm on the eyelids and eyelid margins. Periodic removal of debris reduces inflammation and stabilizes the tear film on the eyelid margins.

The present apparatus includes a body having a finger contact region and an eyelid, contact region. A pair of pads can be integrally formed within the eyelid contact region. Two groups of first bristles can be spaced from each other and can extend a first length from the eyelid contact region from points between the pair of pads. A group of second bristles can extend a second length from the eyelid contact region greater than the first length, and can also extend from points between the two groups of first bristles.

The apparatus includes an eyelid contact region that can be brought to bear against an eyelid, wherein the eyelid can comprise an eyelid margin and eyelashes. The eyelid contact region can be brought to bear against the upper eyelid, the lower eyelid, or both simultaneously. The eyelid contact region comprises a brush that exfoliates and provides cleaning and removal of biofilm, keratinized epithelium, dead skin, and any blockage within and/or on the upper eyelid, lower eyelid, or both.

The body of the apparatus includes not only the eyelid contact region, but also a finger contact region. According to one embodiment, the finger contact region comprises an opening in an elongated sleeve to accommodate a finger of a user. Upon user movement of his or, her finger, the eyelid contact region will then correspondingly move to massage an eyelid of that user. According to an alternative embodiment, the, finger contact region comprises an extension. The extension can be integral to the body and can extend from the body to accommodate two fingers of a user applied on opposite sides of the extension. The user, upon movement of his or her two fingers, causes the eyelid contact region to massage an eyelid of that user.

According to one embodiment, the exfoliation brush on the eyelid contact region of the apparatus can include first bristles and second bristles. There can be two groups of first bristles spaced from each other and extending a first length from the eyelid contact region and between a pair of pads. A group of second bristles can also extend from the eyelid contact, region but will extend a second length greater than the first length. The group of second bristles extend from the eyelid contact region and from points that exist between the two groups of first bristles. Accordingly, the group of second bristles exists between the two groups of first bristles, and are longer than the first bristles. By forming the group of second bristles longer than the first bristles, the second bristles are brought to bear against the eyelashes and eyelid margin of the eyelid of a user more so than the two groups of first bristles. Moreover, if the eyelid contact region and corresponding first and second bristles are brought to bear against one or both eyelids, the distal regions of the eyelid will primarily receive the second bristles more so than the first bristles. Because the second bristles are longer than the first bristles, the distal regions of the eyelid or eyelids receives more exfoliation force and thus greater treatment at those distal regions where removal of debris is more critical. It is in the distal regions that the biofilm can build up and blockage can occur.

According to another embodiment, a method is provided for removing debris from an eyelid. The method includes, contacting at least on finger of a user onto a finger contact region of a body. Thereafter, frictional force is applied to the eyelid by moving the at least one finger in a massaging motion to correspondingly move an eyelid contact region of the body. Greater frictional force is applied upon the eyelid margin region and eyelashes of the eyelid than other regions of the eyelid.

The eyelid contact region is configured to contact an eyelid of the user, whereas the finger contact region is configured to contact a finger of that user. Contacting at least one finger comprises inserting one finger into an opening within an elongated sleeve. Alternatively, contacting at least one finger comprises contacting two fingers on opposite sides of an extension. Applying greater frictional force comprises pressing with greater force a group of second bristles upon a distal region of the eyelid than the force at which at least one of two groups of first bristles is applied further from the distal region. The distal region preferably comprises the eyelid margin region and the eyelashes where the meibomian gland and tear ducts exists. The distal region comprises the eyelid margin region and the eyelashes on the upper eyelid, the lower eyelid and both upper and lower eyelids.

Embodiments described herein comprise a combination of features and characteristics intended to address various shortcomings associated with certain prior devices, systems and methods. The foregoing has outlined rather broadly the features and technical characteristics of the disclosed embodiments in order that the detailed description that follows may be better understood. The various characteristics and features described above, as well as others, will be readily apparent of those skilled in the art upon reading the following detailed description, and by referring to the accompanied drawings.

It should be appreciated that the conception and the specific embodiments disclosed may be readily utilized as a basis for modifying or designing other structures or for carrying out the same purposes as the disclosed embodiments. It should also be realized that such equivalent constructions do not depart from the spirit and the scope of the principles disclosed herein. The objects and advantages of the concepts may be realized and obtained by means of the methodologies, components and combinations strictly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a front isometric view of the apparatus according to one embodiment;

FIG. 2 is a top isometric view of the apparatus of FIG. 1;

FIG. 3 is a left top and side isometric view of the apparatus of FIG. 1;

FIG. 4 is a side plan view of the apparatus of FIG. 1;

FIG. 5 is a right top and side isometric view of the apparatus of FIG. 1;

FIG. 6 is a front isometric view of the apparatus according to another embodiment;

FIG. 7 is a top isometric view of the apparatus of FIG. 6;

FIG. 8 is a left top and side isometric view of the apparatus of FIG. 6

FIG. 9 is a side plan view of the apparatus of FIG. 6;

FIG. 10 is aright top and side isometric view of the apparatus of FIG. 6;

FIG. 11 is a side view of the apparatus of FIGS. 1-5 self-administered and applied by a user to an eyelid of that user; and

FIG. 12 is a side view of the apparatus of FIGS. 6-10 self-administered and applied by a user to an eyelid of that user.

### DETAILED DESCRIPTION

The following discussion is directed to various exemplary embodiments. However, one of ordinary skill in the art will understand that the examples disclosed herein have broad application, and the discussion of any embodiment is met only to be exemplary of that embodiment, and not intended to suggest that the scope and the disclosure, including the claims, is limited to that embodiment.

The drawing figures are not necessarily to scale. Certain features and components herein may be shown exaggerated in scale or in somewhat schematic form and some detail of conventional elements may not be shown in interest of clarity and conciseness.

In the following discussion and in the claims, the term “including”, “comprising” and “having” are used in an open-ended fashion, and thus should be interpreted to mean “including, but not limited to . . . ” Also, the term “coupled” or “couples” is intended to mean either an indirect or direct connection. In addition, as used herein, the term “axial” and “axially” generally mean along or parallel to a given axis (e.g., x, y or z direction or central axis of a body, opening or port). The terms “radial” and “radially” generally mean perpendicular to the given axis.

FIG. 1 illustrates an apparatus 10 that can be self-administered by a user on the user's eyelid (including each eyelid in sequence or both simultaneously). Apparatus 10 includes a body 12 having a finger contact region 14 and an eyelid contact region 16.

According to one embodiment, the finger contact region 14 comprises an opening 18 in an elongated sleeve. Opening 18 is shown in phantom extended into body 12 to accommodate a finger 20. In the example embodiment of FIG. 1, the sleeve of body 12 comprises a flexible, elastomeric material and can be semi-tubular with a closed end opposite the opening 18. The sleeve outer surface can be tapered radially outward toward the closed end. Conversely, the sleeve inner surface shown in phantom can be tapered radially inward toward the closed end.

According to the example of FIG. 1, the sleeve of body 12 is used to cover a fingertip of a user and tapers from the opening 18 to the narrower closed end to maintain the fingertip inside the sleeve. Because the sleeve of body 12 is flexible and elastic, body 12 not only moves in the direction of which finger 20 directs it, but body 12 can bend or flex if the fingertip bends or flexes.

Configured near the closed end of the sleeve is the eyelid contact region 16, which comprises bristles of a brush. The bristles are integrally formed as part of body 12 and extend outward from the eyelid contact region 16 at varying lengths or distances therefrom. The bristles are preferably made of the same material as body 12, and therefore are formed concurrently with body 12, all at the same time, as a unibody structure. The bristles can therefore be made elastic and flexible, similar to the body 12. When eyelid contact region is brought near and contacts with an eyelid of the user, the bristles can deform. When finger 20 moves, the bristles can correspondingly move in a massaging motion to remove debris from the eyelid.

The bristles of the brush within eyelid contact region 16 can be grouped according to their length. For example, two groups of first bristles 24a and 24b are formed around a group of second bristles 26. Referring to the example front isometric view of FIG. 1 and the top isometric view of FIG. 2 in combination, a better understanding of the arrangement of the two groups of first bristles 24a, 24b, and a group of second bristles 26 is provided. As illustrated, in FIGS. 1 and 2, the group of second bristles 26 extend a second length from the, eyelid contact region 16. The second length is greater than the first length at which the two groups of first bristles 24a and 24b extend. The two groups of first bristles 24a, 24b can have differing numbers of bristles in each group, or the same number of bristles. The two groups of first bristles 24 can also contain first bristles that are of differing lengths extending from the eyelid contact region 16, or the same length. Similarly, the second bristles 26 can extend the same length or differing lengths from each other. However, regardless of whether the individual second bristles extend at the same or differing lengths, their lengths on average or individually are greater than the first lengths at which the individual first bristles extend a differing amount from each other or the same amount as one another.

In addition to the first and second groups of bristles, a pair of pads 28a, 28b area arranged on the outward tapered outer surface closed end of the body 12. Pads 28a, 28b are integrally formed within the eyelid contact region 16. Pads 28a and 28b are better illustrated in the top view of the outward tapered closed end of FIG. 2, and are positioned on opposite sides of the bristles that form the groups of first and second bristles 24a, 24b and 26. Preferably, the pair of pads 28a, 28b comprise a pair of substantially smooth pad surfaces that are coplanar to each other and on opposite sides of bristles 24a, 24b, and 26. The substantially smooth planar surface of pads 28a, 28b are absent any bristles, and but may be uneven with one or more ridges extending from the pad surface if desired.

According to the embodiment of FIGS. 1 and 2, the pair of pads 28a. 28b formed within the eyelid contact region 16 allow for two groups of first bristles 24a, 24b to be spaced from each other. The two groups of first bristles 24a, 24b extend from corresponding points on eyelid contact region 16 between the pair of pads 28a, 28b. A group of second bristles 26 can extend from eyelid contact region 16 and the point of extension is between the two groups of first bristles 24a, 24b. The first 24a of the two groups of first bristles 24a, 24b is spaced from the second 24b of the two groups of first bristles 24a, 24b. Each bristle of the first 24a of the two groups of first bristles 24a, 24b is spaced from each other, and each bristle of the second 24b of the two groups of first bristles 24a, 24b is spaced from each other. Each of the two groups of first bristles 24a, 24b and the group of second bristles 26 is integrally formed as part of the same mold of extruded elastomeric material as body 12 and can project from the eyelid contact region 16 as a unibody structure.

Turning now to FIGS. 3, 4 and 5, a top left and side isometric view, a side plan view, and right top and side isometric view of the apparatus of FIGS. 1 and 2 are shown. The groups of first and second bristles 24a, 24b and 26 are also shown, wherein the lengths of the first bristles can be the same or different from one another, and the lengths of the second bristles can be the same or different from one another. Regardless of their similarity or difference in lengths, the groups of first bristles 24a, 24b extend a first length that is less than the second length at which the group of second bristles 26 extend.

Opening 18 is shown into which a user's finger can be inserted. The finger is preferably inserted into opening 18 so that the eyelid contact region 16 is opposite the fingernail of the user's inserted finger 20. Any flexure of the knuckle of finger 20 can therefore bring the groups of first and second bristles 24a, 24b and 26 forward and upon an eyelid of the user. The eyelid contact region 16, and bristles thereon, conform to the outer contour of a user's eye. This helps to maximize the apparatus effectiveness in removing debris along the eyelid margins and eyelashes. The pads 28a, 28b are preferably positioned on the outer surface of the eyelid proximate the meibomian glands. The position and the softness of the pads 28a, 28b allows for gentle massaging of the glands. The apparatus is to be worked on the finger of a person while cleaning/cleansing the person's upper and lower eyelashes, eyelids, and eyelid margins.

The eyelid preferably comprises an eyelid margin and eyelashes. The eyelid contact region 16 is shown having a shape that substantially matched and conforms to an outer shape of the eyelid margin and eyelashes. The pair of pads 28a, 28b (FIG. 2) engage with the eyelid margin, and the two groups of first bristles engage with the eyelid margin and eyelashes less so than the group of second bristles 26 and apparatus 10. The reason that the group of second bristles 26 frictionally engage with the eyelid margin and eyelashes more so than the first bristles is because the group of second bristles 26 extend at a greater length than the first bristles. Whenever the user brings apparatus 10 towards the user's eyelid, the group of second bristles 26 will engage with more force onto the eyelid than the first bristles. By placing the group of second bristles 26 near a central region of eyelid contact region 16 and between the two groups of first bristles 24a and 24b, the user can more easily self-administer the appropriate bristle, and bristle length, at greater frictional force upon a distal region of the eyelid. The distal region being the eyelid margin and eyelashes that are more likely to have clogged or obstructed gland openings. Applying the appropriate sized bristle to the appropriate location on the gland openings with precision afforded this disclosure provides a more effective massage treatment of the meibomian glands and tear ducts of the eyelid.

Turning now to FIGS. 6 and 7, apparatus 10 is shown according to an alternative embodiment. Apparatus 10 of FIGS. 6 and 7, like apparatus 10 in FIGS. 1-5, can be self-administered by a user. However, instead of body 12 in FIG. 6 having an opening in an elongated sleeve, body 12 comprises an extension 30 configured to accommodate two fingers 32a and 32b (FIGS. 7-10) applied on opposite sides of extension 30.

The embodiment shown in FIGS. 6-10 therefore includes a body 12 and a pair of pads 28a, 28b formed within the eyelid contact region 16. Also included within eyelid contact region are first and second bristles 24a, 24b and 26. The embodiment shown in FIGS. 6-10 is therefore similar to the embodiment of FIGS. 1-5 with respect to the pads 28a, 28b, first and second groups of bristles 24a, 25b, and 26, as well as the manufacture arrangement and dimensions thereof. A difference between the two embodiments is that the finger contact region 14 (FIGS. 8-10) in the second embodiment does not comprise an opening in an elongated sleeve. Instead, an extension 30 is shown to accommodate two fingers on 32a, 32b on opposite sides of extension 30. The two fingers are preferably two fingers on the same hand of the user, and more preferably the distal ends of the index finger and thumb. Movement of the index finger and thumb causes a massaging movement of the eyelid contact region 16, as well as the corresponding bristles formed thereon, across an eyelid and specifically the eyelid margin and eyelashes of the user.

Turning now to FIG. 11, the apparatus 10 according to the embodiment of FIGS. 1-5 is shown applied to one or both eyelids 40a, 40b of a user. Eyelids 40a, 40b can be the upper and lower eyelids surrounding an eye 42 of the user. At a distal region of each eyelid 40a, 40b are eyelashes 44, as well as duct openings for the tear ducts or the meibomian glands. Such ducts or glands being responsible for the production of the ocular tear film. Since the glands can become blocked, plugged of obstructed, apparatus 10 connotes a shorter first bristles 24a, 24b and longer second bristles 26 brought to bear against the distal regions of eyelid 40a, 40b. The second bristles 26 can be placed with greater force upon the distal regions of the eyelid margins as shown by the force vector 46. Finger 20 within opening 18 of the first embodiment moves the apparatus 10, and specifically bristles 24a, 24b and 26 in three dimensions (x,y and z). Bristles 24a, 24b, and 26 can be moved in a massaging pattern across the distal regions of an upper eyelid 40a, a lower eyelid 40b, or simultaneously across both eyelids 40a and 40b.

FIG. 12 illustrates apparatus 10 according to the second embodiment, wherein two fingers 32a, 32b are used to press against opposing surfaces of extension 30, and to correspondingly move in three dimensions bristles 24a, 24b and 26 across one or both distal regions of upper and lower eyelids 40a and 40b. In accordance with the arrangement shown in FIGS. 11 and 12, a method is provided for removing debris from an eyelid (upper eyelid 40a, lower eyelid 40b, or simultaneously both eyelids 40a and 40b). The method includes contacting at least one finger of a user onto a finger contact region of a body of apparatus 10. Frictional force is then applied to the eyelid 40a and/or 40b by moving the at least one finger in a massaging motion to correspondingly move an eyelid contact region 16 of the body. As force is applied to the eyelid, greater frictional force is applied upon the eyelid margin region and eyelashes than other regions of the eyelid. The greater frictional force is a result of the group of second bristles 26 being longer than the group of first bristles and therefore more readily contacting via force 46 the distal region of the eyelid 40a, 40b.

As shown in the FIGS. 1-12, apparatus 10 can be self-administered for treating blepharitis, MGD and DED using different arrangements of apparatus 10 applied to select regions of the upper 40a, lower 40b, or both eyelids 40a, 40b. Pads 28a and 28b are also applied to the distal regions of eyelids 40a, 40b. Since pads 28a and 28b are relatively elastic and flexible, pads 28a, 28b allow for gentle massaging of the glands and tear duct openings. Apparatus 10 therefore is self-administered to work on the finger or fingers of the user while cleaning and cleansing the user's upper and lower eyelashes, eyelids and eyelid margins.

While exemplary embodiments have been shown and described, modifications thereof can be made by one skilled in the art without departing from the scope or teachings herein. The embodiments described herein are exemplary only and are not limiting. Many variations and modifications of the apparatus and method described herein are possible and are within the scope of the disclosure. For example, the body, finger contact region and eyelid contact region can have different shapes and can be formed as different integral body parts. The taper according to the first embodiment can vary, the number of fingers inserted into opening 18 can vary, the extension 30 in the second embodiment can vary as well as the location at which the extension protrudes from the top of the apparatus can vary. The grouping and lengths of the first and second bristles 24a, 24b and 26, the number of bristles in each group and the lengths of individual bristles can vary provided a set of one or more bristles can be made longer than the other sets within other groups so that longer bristles can be readily and more precisely directed to the specific region needing the massage. Unless it is stated otherwise, the steps in a method claim may be performed in any order unless a method claims specifies an intended order.

It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

What is claimed is:

1. An apparatus for removing debris from an eyelid, comprising:
a body having a finger contact region and an eyelid contact region;
a pair of pads integrally formed within the eyelid contact region;
two groups of first bristles spaced from each other and extending a first length from the eyelid contact region and between the pair of pads; and
a group of second bristles extending a second length from the eyelid, contact region greater than the first length and between the two groups of first bristles.

2. The apparatus of claim 1, wherein the first of the two groups of first bristles is spaced from the second of the two groups of first bristles. wherein each bristle of the first of the two groups of first bristles is spaced from each other, and wherein each bristle of the second of the two groups of first bristles is spaced from each other.

3. The apparatus of claim. 1, wherein the group of second bristles is spaced from each of the two groups of first bristles and from each other.

4. The apparatus of claim 1, wherein each of the two groups of first bristles and the group of second bristles are integrally formed as part of the eyelid contact region and project from the eyelid contact region.

5. The apparatus of claim 1, wherein the eyelid comprises an eyelid margin and eyelashes, and wherein the eyelid contact region comprises a shape that substantially matches and conforms to an outer shape of the eyelid margin and eyelashes.

6. The apparatus of claim 5, wherein the pair of pads are configured to frictionally engage with the eyelid margin, and the two groups of first bristles are configured to frictionally engage with the eyelid margin and eyelashes less so than the group of second bristles when the apparatus is self-administered and in use by a user upon that user's eyelid.

7. The apparatus of claim 1, wherein the finger contact region comprises an opening in an elongated sleeve to accommodate a finger of a user, and wherein the user upon movement of the finger causes the eyelid contact region to massage an eyelid of the user.

8. The apparatus of claim 7, wherein the sleeve comprises a flexible, elastomeric material.

9. The apparatus of claim 7, wherein the sleeve is semi-tubular with a closed end opposite the opening and tapered toward the closed end.

10. The apparatus of claim 1, wherein the finger contact region comprises, an extension configured to accommodate two fingers of a user applied on opposite sides of the extension, and wherein the user, upon movement of the two fingers, causes the eyelid contact region to massage an eyelid of the user.

11. The apparatus of claim 1, wherein the pair of pads comprise a corresponding pair of substantially smooth pad surfaces coplanar to each other and absent any bristles.

12. A self-administered apparatus for removing debris from an eyelid, comprising:
a body having a finger contact region and an eyelid contact region;
a pair of pads integrally, formed within the eyelid contact region and brought to bear against an eyelid margin of the eyelid of a user;
two groups of first bristles spaced from each other and extending a first length from the eyelid contact region and between the pair of pads; and
a group of second bristles extending a second length from the eyelid contact region greater than the first length and between the two groups of first bristles, wherein the group of second bristles are brought to bear against eyelashes and the eyelid margin of the eyelid of a user more so than the two groups of first bristles.

13. The apparatus of claim 12, wherein each of the two groups of first bristles and the group of second bristles are integrally formed as part of the eyelid contact region and project from the eyelid contact region.

14. The apparatus of claim 12, wherein the group of second bristles are brought to bear against a central area adjoining a pair of eyelids, and wherein the two groups of first bristles are brought to bear, to a lesser extent than the group of second bristles, against two areas on opposite sides of the central area adjoining the pair of eyelids.

15. The apparatus of claim 12, wherein the group of second bristles and the two groups of first bristles are brought to bear against upper and lower eyelid margins and eyelashes of the user when at least one finger of the user is applied to the finger contact region and moved.

16. A method for removing debris from an eyelid, comprising:
contacting at least one finger of a user onto a finger contact region of a body;
applying frictional force to the eyelid by moving the at least one finger in a massaging motion to correspondingly move an eyelid contact region of the body; and
applying greater frictional force upon an eyelid margin region and eyelashes of the eyelid than other regions of the eyelid.

17. The method of claim 16, wherein the contacting comprises inserting one finger into an opening within an elongated sleeve.

18. The method of claim 16, wherein the contacting comprises contacting two, fingers on opposite sides of an extension.

19. The method of claim 16, wherein the applying greater frictional force comprises pressing with greater force a group of second bristles upon a distal region of the eyelid than pressing with lesser force at least one of two groups of first bristles further from the distal region.

20. The method of claim 19, wherein the distal region comprises the eyelid margin region and the eyelashes.
